(19) United States
(12) Patent Application Publication
(10) Pub. No.: US 2002/0015727 A1
(43) Pub. Date: Feb. 7, 2002

(54) HAIR COMPOSITION USED FOR OBTAINING IMPROVED OPTICAL GLOSS

(76) Inventors: RITA HATFIELD, GLEN ELLYN, IL (US); JOANNE CRUDELE, WAUCONDA, IL (US); ANTONIO LOPEZ, HANOVER PARK, IL (US); EUGENE ZEFFREN, CHICAGO, IL (US); RICHARD MARK BRUCKS, CHICAGO, IL (US)

Correspondence Address: UNILEVER, PATENT DEPARTMENT, 45 RIVER ROAD, EDGEWATER, NJ, 07020, US

(21) Appl. No.: 09/290,517

(22) Filed: Apr. 12, 1999

(51) Int. Cl. A61K 9/127

(52) U.S. Cl. 424/450

(57) ABSTRACT

The invention relates to hair care compositions which comprise liposomes that contain 19-methyleicosanoic acid (19-MEA), an emulsion that contains 19 MEA, and a dispersion of liposomes containing 19-MEA in an emulsion. More specifically, we have now found that liposomes or nonionic surfactant vesicles (nsv's or NSV's) can be formed which are storage-stable and possess an affinity for hair. When included in a hair treatment composition, these liposomes deposit on the hair and some liposomes or liposomal fragments remain on the hair during rinsing or if not rinsed-off, such as, with a leave-on composition, and enhance the deposition on the hair of active ingredient 19-MEA with which they are combined.

### FIELD OF THE INVENTION

This invention concerns processes and compositions for the treatment of human hair. More particularly the invention concerns hair care compositions that provide optimal deposition of lipid active ingredients to improve the conditioning of the hair and to improve the gloss of the hair.

### BACKGROUND OF THE INVENTION AND PRIOR ART

The use of nonionic surfactant vesicles is not unique. In fact there are patents covering their preparation and composition, hereby incorporated by as referenced. Additionally, methyl branched fatty acids, or their salts, esters or amides are reported to increase shine or gloss. While 19-MEA is a branched methyl fatty acid, an examination of the structures claimed in various patents reveals that the branch of interest is the 19th carbon for c20 straight chain (i.e. 18-methyleicosanoic acid) or, if the chain is longer, one end of the branch is ethyl (i.e. CH2CH3). It is the benefits observed for hair shine when nonionic vesicles are used as a rinse-off topical agent that is novel and unexpected. In addition, the more specific claim is the use of nonionic vesicles to deliver 19-MEA which consistently and significantly increases the optical gloss factor of hair as measured by image analysis. Thus, the combination of 19-MEA with nonionic vesicles provides a unique shine benefit to hair never before measured or observed with other hair care compositions.

Publications which relate to this field of technology are as follows:

1. Waranuch et al “Controlled Topical Drug Delivery . . . ” Proceeding Int'l Symposium controlled Rel. Bioactive Materials Vol.24, 1997, pp. 841-842;

2. U.S. Pat. No. 4,911,928, Wallach et al;

3. U.S. Pat. No. 4,855,090; Wallach et al;

4. U.S. Pat. No. 4,895,452 Yiuornas et al;

5. U.S. Pat. No. 5,665,380, Wallach et al;

6. U.S. Pat. No. 4,643,600, Wallach et al; and

7. U.S. Pat. No. 4,605,704, Wallach et al.

### BRIEF SUMMARY OF THE INVENTION

The invention relates to hair care compositions which comprise liposomes that contain 19-methyleicosanoic acid (19-MEA), an emulsion that contains 19 MEA, and a dispersion of liposomes containing 19-MEA in an emulsion. More specifically, we have now found that liposomes or nonionic surfactant vesicles (nsvs or NSVs) can be formed which when storage-stable, possess an affinity for hair. When included in a hair treatment composition, these liposomes deposit on the hair and liposomes or liposomal fragments remain on the hair following rinsing or if not rinsed-off, such as, with a leave-on composition, and enhance the deposition on the hair of active ingredient 19-MEA with which they are combined.

Accordingly, in a first aspect, the present invention provides a process for the deposition of 19-MEA on hair by nonionic surfactant vesicles.

In a second aspect, the present invention provides a composition comprising nsv's and an active ingredient, which when applied to hair will cause enhanced deposition of the active ingredient on the hair than previously achievable. More specifically, nsv's contain 19-methyleicosanoic acid (19-MEA) which when delivered to the surface of the hair fiber elicit a statistically significant (95% confidence index, LSD) increase in optical gloss of hair arrays by reducing the amount of diffuse reflection on the surface of the hair and thus on the hair array. The invention also provides for compositions which are emulsions containing 19-MEA and dispersions of nsvs containing 19-MEA in an emulsion. Such compositions when applied to hair also provide an increase in optical gloss of hair arrays. The compositions of the invention also provide conditioning benefits to the hair.

### DETAILED DESCRIPTION OF THE INVENTION

Unless other indicated, as used herein % means weight %. Starting materials set forth herein are either known or can be prepared in accordance with known methods.

As used herein the terms liposomes and nonionic surfactant vesicles (nsv's) are interchangeable.

Rinse-off compositions of the invention include rinse-off conditioners.

Leave-on compositions of the invention include conditioners, solutions, sprays, gels, mousses, hair masques, pomades, and serums.

A preferred process according to the invention comprises the following steps:

(a) forming a dispersion of nsv's incorporating 19-MEA;

(b) processing the dispersion into a hair treatment composition; and

(c) treating the hair with the composition.

Alternatively, treating the hair with the product of step (a), that is a dispersion of nsvs incorporating 19-MEA alone, provides optical gloss and conditioning benefits to the hair.

We have found that the dispersion of step (a) may be prepared by melting the surfactants and 19-MEA and vortex-mixing or homogenizing or sonicating with an aqueous solution with methods set forth in the Wallach patent, which is hereby incorporated by reference or forming a film of surfactants and adding buffer to the film with shaking in accordance classical thin film hydration methods.

The particle sizes are formed in a broad range of about less than 1 micrometer to about 15 micrometers in diameter and can be visualized using conventional contrast microscopy techniques. Additionally, the nsv's may be sized down by several methods known in the art such as sonication, extrusion, and microfluidization. This induces the formation of smaller and more uniform nsvs and the structures so generated may be easily visualized and sized by transmission electron microscopy or sized by laser light scattering techniques.

More specifically, 19-MEA was incorporated into nonionic surfactant vesicles. A multilamellar nsv system was prepared which contained glycerol dilaurate, cholesterol and polyoxyethylene (10) stearyl ether in a weight percent ratio of about 45:15:40 respectively in a manner similar to that reported in Waranuch et al., (1996) “Controlled Topical Delivery of Cyclosporin-A from Nonionic Liposomal Formulations”. Proceed Intern. Symp. Control. Rel. Bioact. Mater. 23 Pages 327-328. This latter publication is hereby incorporated by reference. The 19-MEA is incorporated into the vesicles at a concentration of about 0.001 mg/ml to about 5 mg/ml, more preferably from about .01 mg/ml to about 2 mg/ml.

It is preferable to optimize encapsulation to allow the best efficiency of deposition from the liposomal dispersions of the invention, although the exact mechanism by which this occurs is unclear. In general, it is less preferable to add the active ingredient at a later stage than during liposome formation.

Compositions according to the present invention may comprise a dispersion of nsvs, in which the concentration of cholesterol added during processing (of the weight percent of total lipid in the hair treatment composition) is suitable from about .001% to about 25% and the concentration of nonionic surfactant added during processing is suitable from about .001% to about 58% glyceryl dilaurate and from about 1.0% to about 84% polyoxyethylene (10) stearyl ether and from about .001% to about 5.0% 19-MEA, preferably from about 0.2% to about 2.0% 19-MEA, of the weight percent of total lipid in the hair treatment composition.

Examples of suitable nonionic surfactants to prepare nsvs or emulsions include: polyoxyethylene surfactants, glycerol esters (2-3 chain preferably), sorbitan derivatives, polyglycerol derivatives, polyethylene glycol esters, fatty acids, and other sterols.

Other optional ingredients of the invention which may be contained in the nsv's include sunscreens and anti-dandruff agents as outlined in U.S. Pat. No. 5,605,704, issued Feb. 25, 1997, which is hereby incorporated by reference.

Other optional active ingredients are vitamin E and ceramides, or derivatives thereof.

Compositions according to the present invention may further comprise one or more optional ingredients which are normally found in hair treatment compositions. The compositions of the invention will preferably take the form of post-wash hair conditioning compositions or hair treatment masques, but may also take the form of hair styling compositions or the like.

As further optional components for inclusion in the compositions of the invention, in addition to water, the following may be mentioned: pH adjusting agents, viscosity modifiers, pearlescers, opacifiers, suspending agents, preservatives, colouring agents, dyes, proteins, herb and plant extracts, polyols, other moisturizing and/or conditioning agents, fragrance, monovalent, divalent, and trivalent salts, and antioxidants. 19-MEA may also be incorporated into emulsions. Compositions of the invention include such emulsions. Methods of the invention include the application to hair of such emulsions for the purpose of increasing the optical gloss of hair arrays and for the purpose of conditioning the hair.

Compositions of the invention which are emulsions are prepared as follows:

The oil, nonionic surfactants and 19-MEA are placed in a suitable vessel and heated (60-90° C.) with mixing until all the components are melted. Next, the water phase is heated to the same temperature as the oil phase. When the water phase and oil phase are at an equivalent temperature the water is slowly added to the oil phase with mixing. Once all the water is added to the oil, the mixture is allowed to cool to room temperature, with mixing. The emulsion is allowed to equilibrate for one day prior to use. There are many methods, known to the art, for the processing of emulsions which could be applied to the preparation of the emulsion described.

Compositions according to the present invention may comprise a dispersion of nsvs in an emulsion.

Embodiments of the present invention will now be further illustrated by reference to the following examples. All amounts given are in % by weight, unless other stated.

| Ingredient | Concentration (Weight Percent) |
|---|---|
| Glycerol dilaurate** | 2.25% |
| Cholesterol** | 0.75% |
| Polyoxyethylene(10) stearyl ether** | 2.0% |
| 19-MEA | 0.2% |
| Solubilized in chloroform, evaporation | |
| of chloroform, thin film formation | |
| Phosphate Buffer | 94.8% |

**GDL, CH and POE are present in a ratio of 45:15:40 in a total lipid concentration of 50 mg/ml.

| Ingredient | Concentration (Weight Percent) |
|---|---|
| Glyceryl Dilaurate | 0.5% |
| Polyoxyethylene (10) Stearyl Ether | 2% |
| Mineral Oil (65/75) | 5% |
| 19-MEA | 0.2% |
| Water | Qs |

| Ingredient | Concentration (Weight Percent) |
|---|---|
| Mixture from Example A | 80% |
| Mineral Oil (65/75) | 20% |

### Measurement of Hair Fiber Array Optical Gloss/shine by Color Image Analysis

A shine factor S_{F} is calculated from a variety of parameters including diffuse reflectance, specular reflectance, and shape of the specular reflectance band/curve, measured by color image analysis technique. Each treated tress is wrapped and clamped onto a large non-reflective cylinder. Each tress is illuminated at an incident angle of 30 degrees with a high frequency light (5400° K, color rendition > 98, intensity 1400 lux) while a video camera mounted perpendicular to the longitudinal axis of the cylinder captures the relevant highlighted and dark regions of the tress. The captured image is then digitized and resolved into its primary color components: red, green, and blue channels.

Shine measurements are performed within an area bounded by a rectangular frame measuring 400×60 pixels. Within each bounded area the light intensity distribution for each primary color is measured. A shine factor (S_{F}) which is dependent on two groups of parameters, one related to the intensities of specular and diffuse reflection(S/D) and the other (H/W ½) characterizing the shape of the specular reflection maximum, is calculated for each color component of a tress [S_{F}=(S/D) (H/W ½)]. Statistical analysis is performed using Statgraphics and Fisher's least significant difference (LSD) procedure to discriminate among the means in multiple comparison (Ref. 4 and 5 which are hereby incorporated by reference.

### Hair Optical Gloss/Shine Results

Results obtained in a series of two experiments are contained in Table I, FIG I, and Table II, FIG II (Ref. 5 and 6). In both experiments, a variety of vesicle preparations, emulsions and benchmark commercial products were analyzed following topical application on hair for the potential of increasing the overall optical gloss or shine of hair. Some of the same vesicle preparations were used in both experiments. Together the two experiments were designed to test the effect of (1) vesicles (2) vesicle composition (3) nonionic surfactants, (4) 19-MEA, and (5) emulsion formulations on optical gloss and condition of the hair.

### Vesicles

In the first experiment, four different treatment groups, which all comprise hair treated with various vesicle preparations, showed statistically significant increases in shine over the water soaked tress at the 95% confidence level. These treatments are DPPC MLVs 3X, NSVs 1X, NSVs 3X blow dry and NSVs with 19-MEA 3X. After the four vesicle preparations cited above, NSV 3X treatment no blow drying was next in rank order as having a shine factor greater than the water soaked tress, although not statistically greater than the untreated, water soaked control hair.

In a second experiment, as with the first experiment, hair treated with a 3X application and rinse off of NSVs containing 19-MEA had a significantly higher overall shine factor than the untreated control (95% Cl, LSD), or hair treated with NSVs with eicosanoic acid 3X, NSVs-1X, 1% or 2% silicone Organics 1X, NSV-MF (microfluidized), Net (1% silicone) 1X,vinegar rinse 3X, Pantene 2 in 1 (Normal) 1X, 19-MEA deposited in a chloroform base 3X.

As a group, vesicles provide the hair with increased optical gloss/shine. While numerically greater values are obtained for most vesicle treatments, the statistical significance of the results is composition dependent.

### Vesicle Composition

Vesicle compositions tested in the two experiments include dipalmitoylphosphatidycholine, NSVs, NSV with 19-MEA, NSVs with eicosanoic acid (EA) and NSVs with cholesterol sulfate (CS). When comparing three time product applications in both experiments, 19-MEA NSVs treated hair was statistically greater in shine factor than untreated hair. Additionally, of all vesicle compositions tested, hair treated with 19-MEA NSVs ranked the greatest in shine factor. Other vesicle compositions which resulted in statistically greater shine factors than untreated hair in the first experiment were NSVs 1X, DPPC MLVs 3X and NSV 3X with blow dry.

In the second experiment, the only vesicle composition which provided statistically greater shine than the untreated control was NSVs with 19-MEA. Additionally, in the second experiment, there ware differences between 19-MEA vesicle treated hair and other types of vesicle-treated hair. 19-MEA vesicle treated hair was statistically shinier than hair treated with NSVs with eicosanoic acid 3X and NSVs MF.

Vesicles containing 19-MEA elicit statistically greater increases in shine factor than other vesicle compositions demonstrating that 19-MEA incorporated in a nonionic surfactant vesicle provides consistent and superior optical gloss in comparison to other vesicle compositions.

### Nonionic Surfactants

In the second experiment, nonionic surfactants in emulsion and vesicle forms were compared. A nonionic surfactant emulsion containing mineral oil resulted in a statistically greater shine factor than untreated hair. Nonionic vesicles were tested in experiments 1 and 2. In experiment 1, the NSVs provided statistically greater shine than untreated hair. In the second experiment, NSVs produced a high shine factor, but it was not statistically significant at the 95% confidence level. In the second experiment, the formulations which elicited the highest rank order shine values all contained nonionic surfactants. This suggests that nonionic surfactants are good components for shine.

### 19-MEA

19-MEA shine effects were tested in the following vehicles: NSVs, nonionic surfactantimineral oil emulsion and chloroform. As stated several times previously, 19-MEA in the mineral oil emulsion resulted in an increased shine factor which was not however, statistically greater than untreated. In contrast, 19-MEA in chloroform produced a statistically lower shine factor than untreated hair; depositing this lipid from organic solvent leaves a white residue which dulls the hair. This data demonstrates that 19-MEA must be delivered in the appropriate vehicle to increase shine. Nonionic surfactant vesicles appear to be optimal.

These results demonstrate that vesicles (lipid or nonionic surfactant) increase hair shine but that the unique combination of 19-MEA in NSVs consistently provides superior optical gloss, further strengthening the uniqueness of the NSV and 19-MEA relationship.

### Relationship Between Deposition and Shine Parameters

In the first experiment, preliminary evaluation of how the different parameters of the shine measurement contributed to the overall shine factor along with the microscopic evaluation of the treated hair revealed two key and related observations (Ref. 5).

1) That for hair treated with NSVs alone and NSVs with 19-MEA the significant observable change in the parameters of the shine factor measurements compared to the untreated control is reduction in diffuse reflection

2) Microscopic evaluation of hair treated with NSVs and NSVs with 19-MEA revealed a significant smoothing of the cuticle edges and to some extent the face of the cuticle plate which would reduce diffuse scattering at the surface of the hair fibers.

In the second experiment, a variety of commercial shampoos and experimental topical applications were evaluated for hair shine/optical gloss (Ref. 6 which is hereby incorporated by reference). Evaluation of how the different shine parameters contribute to the overall shine factor for the various treatments confirmed key observation 1 of the first experiment, that hair treated with NSVs alone, NSVs in a mineral oil emulsion, or especially with NSVs containing 19-MEA in the vesicle bilayers reduce the diffuse scattering of light from the treated hair fiber array resulting in a higher measurement of overall optical gloss/or shine. In fact the correlation between the measure of overall shine factor (Sᵣ blue component) for all the treatments and the measure of diffuse reflectance (blue light components) is r=.9171 FIG III). Therefore, for the treatments that were evaluated in the first and second experiments, reduction of diffuse reflectance through product deposition or cleansing is the factor in producing the overall increase in shine or optical gloss that is seen with these treatments and also promote fiber smoothness.

The invention relates to the use of nonionic vesicles to deliver 19-MEA which consistently and significantly increases the optical gloss of hair as measured by image analysis. Thus, the combination of 19-MEA with nonionic vesicles provides a unique shine benefit to hair and takes advantage of the superiority of vesicle delivery systems combined with the benefits of a branched fatty acid.

We claim:

1. A rinse-off or leave-in hair treatment composition which comprises:
(A) an oil in water emulsion wherein said oil contains 19-MEA; or
(B) a dispersion of nonionic surfactant vesicles in water, wherein said nonionic surfactant vesicles (nsv) contain 19-MEA; or
(C) a dispersion of nonionic surfactant vesicles containing 19-MEA in an oil in water emulsion.

2. A composition according to claim 1, which is an emulsion and wherein said 19-MEA is present at about 0.01wt. % to about 5.0 wt. %.

3. A composition according to claim 1, which is an emulsion and wherein said 19-MEA is present at about 0.05wt. % to about 2 wt. %.

4. A composition according to claim 1, which is an emulsion and which comprises:

| Ingredient | Concentration (Weight Percent) |
|---|---|
| Glyceryl Dilaurate | 0.5% |
| Polyoxyethylene (10) Stearyl Ether | 2% |
| Mineral Oil (65/75) | 5% |
| 19-MEA | 0.2% |
| Water | Qs |

5. A composition according to claim 1 which is a dispersion of liposomes containing 19-MEA, in water and wherein 19-MEA is present at about 0.01 wt. % of total lipids and surfactants to about 5.0 wt. % of total lipids and surfactants.

6. A composition according to claim 1 which is a dispersion of liposomes containing 19-MEA, in water and wherein 19-MEA is present at about 0.05 wt. % of total lipids and surfactants to about 2.0 wt. % of total lipids and surfactants.

7. A composition according to claim 1 which is a dispersion of liposomes in an oil in water emulsion and wherein 19-MEA is present at about 0.01 wt. % of total lipids and surfactants to about 5.0 wt. % of total lipids and surfactants.

8. A composition according to claim 1 which is a dispersion of liposomes in an oil in water emulsion and wherein 19-MEA is present at about 0.05wt% of total lipids and surfactants to about 2.0 wt. % of total lipids and surfactants.

9. A composition according to claim 1, which is a dispersion of liposomes in water that comprises
(i) glycerol dilaurate at about 0.01 wt. % of total lipids and surfactants to about 58.0 wt. % of total lipids and surfactants;
(ii) cholesterol at about 0 wt. % of total lipids and surfactants to about 15.0 wt. % of total lipids and surfactants; and
(iii) polyoxyethylene (10)stearyl alcohol at about 1.0 wt. % of total lipids and surfactants to about 84.0 wt. % of total lipids and surfactants.

10. A composition according to claim 1, which is a dispersion of liposomes in an oil in water emulsion wherein the dispersion of liposomes is comprised of:
(i) glycerol dilaurate at about 0.01 wt. % of total lipids and surfactants to about 58.0 wt. % of total lipids and surfactants;
(ii) cholesterol at about 0 wt. % of total lipids and surfactants to about 15.0 wt. % of total lipids and surfactants;
(iii) polyoxyethylene (10)stearyl alcohol at about 1.0 wt. % of total lipids and surfactants to about 84.0 wt. % of total lipids and surfactants, and
(iv) an oil in water emulsion.

11. A composition according to claim 9, wherein (i), (ii) and (ii) are present in a weight ratio of about 45:15:40.

12. A composition according to claim 10, wherein (i), (ii) and (ii) are present in a weight ratio of about 45:15:40.

13. A composition according to claim 1, in the form of a rinse-off conditioner.

14. A composition according to claim 1, in the form of a leave-on composition selected from the group consisting of conditioners, solutions, sprays, gels, mousses, hair masques, pomades, and serums.

15. A composition according to claim 1 which is a dispersion of liposomes wherein 19-MEA is present at about 1 mg/ml of total lipids and surfactants to about 5 mg/ml. of total lipids and surfactants.

16. A composition according to claim 1 which is a dispersion of liposomes wherein 19-MEA is present at a concentration of about 2 mg/ml of total lipids and surfactants.

17. A method for increasing the optical gloss of hair arrays which comprises treating said hair arrays with an effective amount of a composition according to claim 1.

18. A method for reducing diffuse reflection from the surface of a hair fiber which comprises treating said surface of the hair fiber with a composition according to claim 1.

19. A method for imparting conditioning benefits to hair which comprises treating said hair with an effective amount of a composition according to claim 1.
